(19) United States
(12) Patent Application Publication
(10) Pub. No.: US 2004/0265440 A1
(43) Pub. Date: Dec. 30, 2004

(54) Food borne pathogen sensor and method

(75) Inventors: Roger Morris, Sebastian, FL (US); Galo Acosta, Sebastian, FL (US); Jerry Hill, Cocoa, FL (US); Alan R. Tank, Bethesda, MD (US); Kyle Newman, Lexington, KY (US); Alan Bishop, Sebastian, FL (US); John A. McMorris III, Indialantic, FL (US)

Correspondence Address: CARL M. NAPOLITANO, PH.D., ALLEN, DYER, DOPPELT, MILBRATH & GILCHRIST, P.A., 255 SOUTH ORANGE AVE., SUITE 1401, P.O. BOX 3791, ORLANDO, FL, 32802-3791, US

(73) Assignee: AgCert International, LLC, Melbourne, FL (US)

(21) Appl. No.: 10/799,312

(22) Filed: Mar. 12, 2004

Related U.S. Application Data
(63) Continuation-in-part of application No. 10/659,222, filed on Sep. 10, 2003.
(60) Provisional application No. 60/411,068, filed on Sep. 16, 2002.
(60) Provisional application No. 60/421,699, filed on Oct. 28, 2002.
(60) Provisional application No. 60/484,869, filed on Jul. 3, 2003.

(51) Int. Cl. G01N 33/02

(52) U.S. Cl. 426/231

(57) ABSTRACT

A sensor for detecting bacteria in a perishable food product includes a gas-permeable material including a pH indicator carried by a housing for placement in a spaced relation to food product or packaging surfaces for effectively detecting a change in carbon dioxide levels within the package. One acid-base pH indicator comprises a mixture of Bromothymol Blue and Methyl Orange with the sensor having an initial green color indicating an alkaline pH of approximately 7.2. The indicator turns orange with a decrease in pH resulting from a presence of carbon dioxide due to bacterial growth, such an indicator reflecting a universally recognizable safe to caution color change.

### CROSS-REFERENCE TO RELATED APPLICATIONS

This application claims priority to pending application having Ser. No. 10/659,222 for “Food-Borne Pathogen and Spoilage Detection Device and Method” having filing date Sep. 10, 2003, which itself claims priority to provisional applications having Ser. No. 60/411,068, filed Sep. 16, 2002, for “Food Borne Pathogen Detection Device and Method for Packaged Meat”; Ser. No. 60/421,699, filed Oct. 28, 2002, for “Food Borne Pathogen Detection Device and Method for Packaged Perishable Foods”; and Ser. No. 60/484,869, filed Jul. 3, 2003, for “Food Borne Pathogen Detection Device and Method,” all commonly owned, the disclosures of which are herein incorporated by reference.

### FIELD OF THE INVENTION

The present invention generally relates to pathogen detection devices and methods, and in particular, to devices and methods for visually detecting food spoilage.

### BACKGROUND OF THE INVENTION

Food borne diseases as well as food spoilage remain a significant burden in the global food supply. In the U.S. alone there are 76 million cases of food-borne illnesses annually, which is equivalent to one in every four Americans, leading to approximately 325,000 hospitalizations and over 5000 deaths annually. According to the GAO and USDA, food-borne pathogens cause economic losses ranging from $7 billion to $37 billion dollars in health care and productivity losses. Hazard Analysis and Critical Control Point (HACCP) regulations state that a hazard analysis on a food product must include food-safety analyses that occur before, during, and after entry into an establishment. There is a clear need to ensure that food transported from the processor to the consumer is as safe as possible prior to consumption. For example, the development of antibiotic resistance in food borne pathogens, the presence of potential toxins, and the use of growth hormones all indicate a need for further development of HACCP procedures to ensure that safer food products are delivered to the consumer. There is also a need to monitor foods being handled by a consumer even after such food is purchased, partially used, and stored for future use.

Meat, for example, is sampled randomly at the processor for food borne pathogens. Generally, no further testing occurs before the meat is consumed, leaving the possibility of unacceptable levels of undetected food-borne pathogens, such as Salmonella spp. and Listeria spp., as well as spoilage bacteria, such as Pseudomonas spp. and Micrococcus spp. being able to multiply to an undesirable level during the packaging, transportation, and display of the product. Subsequently the food product is purchased by the consumer and is transported and stored in uncontrolled conditions that only serve to exacerbate the situation, all these events occurring prior to consumption.

Retailers generally estimate shelf life and thus freshness with a date stamp. This method is inaccurate for two key reasons: First, the actual number of bacteria on the meat at the processor is unknown, and second, the actual time-temperature environment of the package during its shipment to the retailer is unknown. As an example, a temperature increase of less than 3° C. can shorten food shelf life by 50% and cause a significant increase in bacterial growth over time. Indeed, spoilage of food may occur in as little as several hours at 37° C. based on the universally accepted value of a total pathogenic and non-pathogenic bacterial load equal to 1×10⁷ cfu/gram or less on food products. Food safety leaders have identified this level as the maximum acceptable threshold for meat products.

While many shelf-life-sensitive food products are typically processed and packaged at a central location, this has not been true in the meat industry. The recent advent of centralized case-ready packaging as well as “cryovac” packaging for meat products offers an opportunity for the large-scale incorporation of sensors that detect both freshness and the presence of bacteria.

A number of devices are known that have attempted to provide a diagnostic test that reflects either bacterial load or food freshness, including time-temperature indicator devices. To date none of these devices has been widely accepted either in the consumer or retail marketplace, for reasons that are specific to the technology being applied. First, time-temperature devices only provide information about integrated temperature history, not about bacterial growth; thus it is possible, through other means of contamination, to have a high bacterial load on food even though the temperature has been maintained correctly. Wrapping film devices typically require actual contact with the bacteria; if the bacteria are internal to the exterior food surface, then an internally high bacterial load on the food does not activate the sensor. Ammonia sensors typically detect protein breakdown and not carbohydrate breakdown. Since bacteria initially utilize carbohydrates, these sensors have a low sensitivity in most good applications, with the exception of seafood.

Further, known devices and methods for detecting bacteria in food substances typically integrally incorporate the device in to a package at manufacture. Neither the provider nor the consumer is able to continue the monitoring with a repackaging of the food product.

It is desirable to provide a device, food packaging, and associated methods for detecting at least a presence of bacteria in a perishable food product. Further, it is desirable for a consumer to detect a presence of bacteria throughout the handling of the food product by the consumer.

### SUMMARY OF THE INVENTION

The present invention may be directed to detecting at least a presence of bacteria in a perishable food product carried within a container or package prepared by a supplier of the food product or by a consumer handling the food product after purchase. Embodiments of the invention may provide a quantitative measure of bacterial load and detect the presence of bacteria in or on the food product. In addition, a sensor may be safely consumed if mistakenly eaten. A time-temperature capability may also be included in certain embodiments to provide additional information along the food supply chain on any departure from recommended temperature maintenance. Consumer-packaged (cooked or uncooked) foods may also be stored in containers (such as sealable bags or plastic containers) with both bacterial and/or time-temperature sensors providing the consumer with a measure of food freshness and safety.

One sensor of the present invention for detecting a presence of bacteria responsible for food borne illnesses may include a housing having a bore fully extending through the housing and a pH sensitive material carried within the bore. The pH sensitive material includes a pH indicator for providing a visual color change responsive to an increased level of carbon dioxide gas above an ambient level. The indicator detects a change in a gaseous bacterial metabolite concentration that is indicative of bacterial growth, wherein a pH change is affected by a presence of the metabolite. The pH sensitive material is carried within the bore such that opposing first and second surfaces of the material are exposed to an environment within which the housing is to be placed for monitoring and sensing the increased levels of carbon dioxide gas. A fastener is carried by the housing for freely and removably positioning the housing such that the first and second surfaces of the pH sensitive material is in a spaced relation to any adjoining surfaces of food product or container walls within the environment, thus permitting a free movement of the carbon dioxide gas thereabout and direct diffusion of the carbon dioxide gas onto and through the opposing first and second surfaces of the pH sensitive material. Thus gas diffusion on both sides of the pH sensitive material is accomplished, rather than a sensitive surface on only one side, which is typically the case when a sensor is directly attached to a wall of the package material. Again, the space between the sensor and the packaging permits gas to diffuse freely into the pH sensitive material, resulting in a faster detection time.

By way of example, the pH sensitive material, which may includes a mixture of Bromothymol Blue and Methyl Orange, will go through a visual color change from green to orange resulting from the increased level of carbon dioxide gas diffusing through the pH sensitive material for reducing a hydrogen ion concentration and thus reducing the pH. The pH sensitive material may comprise a gel, such as agar, and further may include an antifreeze agent, such as ethylene glycol or glycerol for preventing a freezing of any water component within the gel below 0° C.

By way of further example, the sensor may include the pH sensitive material formed into first and second material portions, each extending between the opposing first and second surfaces. The first material portion may comprise a buffered pH indicator having a reference color. The second material portion may have a recognizable reference color at an initial pH level that changes to a recognizable caution or warning color at a predetermined pH level, wherein the warning color visually contrasts the reference color for alerting a user or consumer. Yet further, the first material portion may include a time-temperature component while the second material portion includes the pH sensitive material, each or both compared to a reference color of a reference material, or a surface of the housing itself.

A thickness dimension of the housing may define the depth or thickness of the bore and a thickness or distance between the first and second opposing surfaces of the pH sensitive material carried within the bore. With such definitions, one preferred ratio of the thickness dimension to an effective width dimension (a diameter in a case of a cylindrical shape) may be in a range of values from 0.003 to 0.3. By way of further example, the pH of the material may range from 7-10 in the ambient level carbon dioxide gas environment.

The sensor may include first and second gas permeable covers carried by the housing for enclosing the pH sensitive material within the bore, and may include gas permeable membranes or covers having holes extending through the covers. The holes may form a descriptive pattern representing a state of the pH sensitive material, by way of example. Further, the covers may have a predetermined color indicative of a pH level for the pH sensitive material, green for safe or orange for caution by way of example. Likewise, the housing may comprise a color representative of an initial color, indicating a safe condition, or a final color, indicating a potentially hazardous condition, for the pH sensitive material. By way of example, the housing may comprise a green color representative of the initial color. A color change from the green color to an orange color may result from the increased level of carbon dioxide gas.

The sensor may include the housing having a handle portion useful in handling the sensor by a user, and a sensor portion having the bore for carrying the pH sensitive material. A fastener useful in attaching the housing may include a tapered handle portion or may carry a pin for piercing a food product carried within a container, or the container itself, within which the food product is to be stored. The fastener may comprise an adhesive material carried by the housing, on the handle portion, by way of example. The adhesive may be of an adhesive tape style, a Velcro material, or the like, for attaching the sensor to an inside container wall while placing the pH sensitive material in a space relation to any nearby surfaces, such as the container wall, the food product, or general food product packaging elements, by way of example. One preferred location for the pH sensitive material is within a lower one-half portion of the container. Further, the housing and the pH indicator may be made of material safe for human consumption.

One aspect of the invention includes a method for detecting a presence of bacteria in a perishable food product. This method comprises the steps of carrying a food product within a package and positioning the sensor within the package. The sensor comprises a pH indicator that is adapted to detect a change in a gaseous bacterial metabolite concentration that is indicative of bacterial growth. A pH change is effected by a presence of the metabolite. The food product and the housing are sealed within the food packaging, and a visual color change of the pH sensitive material is monitored for an indication of a bacterial concentration in the food product in excess of a desired level.

The food product and the sensor may be sealed within a package such that the pH sensitive material of the sensor is spaced away and not directly touching the interior of the package or food product for permitting an improved gas diffusion over known methods and a faster response, thus more desirable for consumer protection.

The features that characterize the invention, both as to organization and method of operation, together with further objects and advantages thereof, will be better understood from the following description used in conjunction with the accompanying drawings. Advantages and improvements of the present invention will become more fully apparent as the following description is read in conjunction with the accompanying drawing.

### BRIEF DESCRIPTION OF THE DRAWINGS

Embodiments of the present invention are herein described with reference to the accompanying drawings, illustrated by way of example and not intended as a definition of the limits of the invention, in which:

FIG. 1 is a top right perspective view of one embodiment of the present invention illustrating a sensor having a housing, wherein a bore within the housing carries a pH sensitive material for viewing a color change thereof;

FIG. 2 is a top plan view of the embodiment of FIG. 1;

FIG. 3 is a right side view of the embodiment of FIG. 1;

FIGS. 4 and 5 are opposing end views of the embodiment of FIG. 1;

FIG. 6 is a partial cross section view illustrating the pH sensitive material, a bacterial growth detector, carried by the housing in a spaced relation to an adjoining food product and container walls;

FIG. 7 is a partial perspective view of one embodiment of a pH sensitive material in combination with a buffered indicator and/or a time-temperature detector;

FIG. 8 is a partial cross section view illustrating an alternate embodiment of the sensor of FIG. 1 including permeable covers for enclosing the pH sensitive material within the bore;

FIG. 9 is a top plan view of one cover embodiment of FIG. 8;

FIGS. 10 and 11 are top plan and side views, respectively, for an alternate embodiment of the sensor of FIG. 1;

FIG. 12 is a partial cross section view illustrating an alternate embodiment of the sensor of FIG. 1;

FIG. 13 is a partial perspective view of the sensor of FIG. 1 positioned within a container;

FIGS. 14A, 14B, and 14C diagrammatically and respectively illustrate the time evolution of bacterial growth detection, with a sensor packaged with a perishable food item; growth of bacterial colonies on the food, the bacteria emitting a gaseous metabolite; and an observable change exhibited by the sensor in response to a decrease in pH;

FIG. 15A is a top, side perspective view of a first embodiment of a bacterial growth detector;

FIG. 15B is a top, side perspective view of a second embodiment of a bacterial growth detector;

FIG. 15C a top, side perspective view of an alternate embodiment of a bacterial growth detector;

FIG. 15D is a top, side perspective view of an alternate embodiment of a bacterial growth detector;

FIG. 15E is a top, side perspective view of an alternate embodiment of a bacterial growth detector;

FIG. 16 is a top, side perspective view of an alternate embodiment of a bacterial growth detector;

FIG. 17 is a top, side perspective view of an alternate embodiment of a bacterial growth detector; and

FIG. 18 illustrates an integrated time-temperature indicator of food freshness.

### DETAIL DESCRIPTION OF THE PREFERRED EMBODIMENTS

The present invention will now be described more fully hereinafter with reference to the accompanying drawings, in which various embodiments of the invention are shown. This invention may, however, be described in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

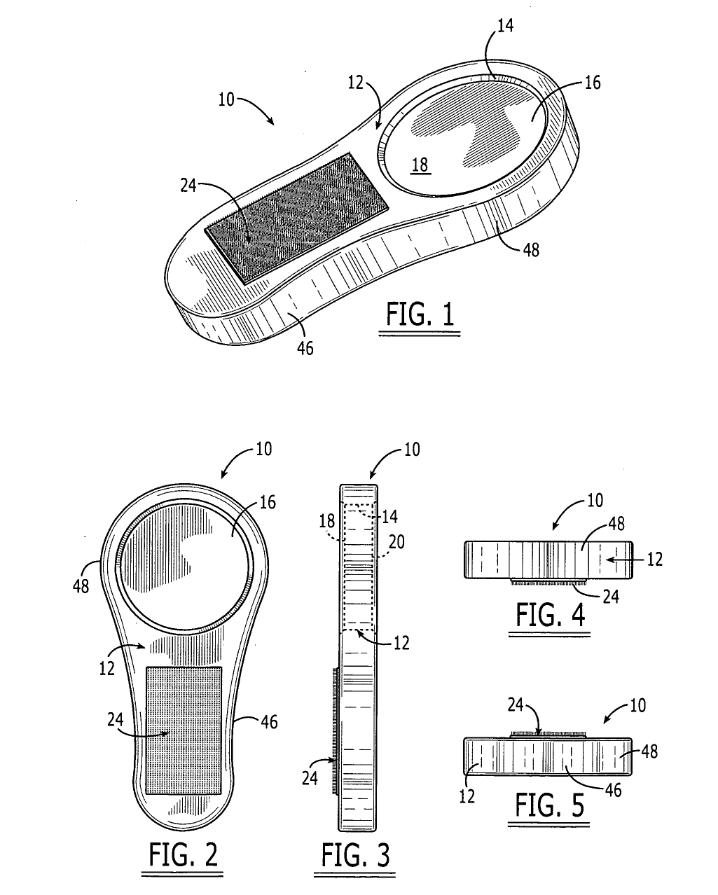

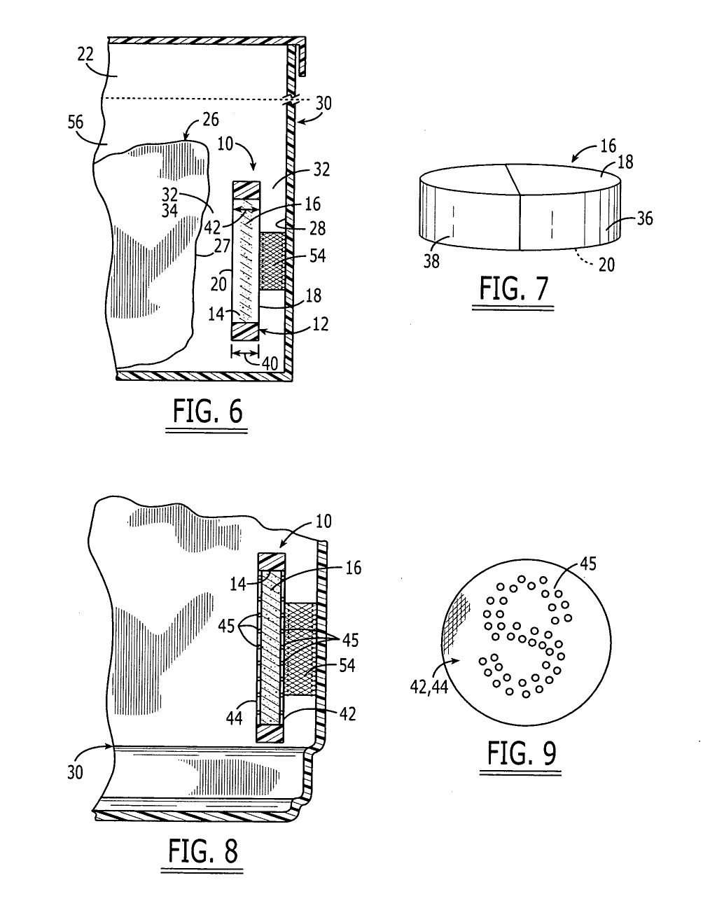

With reference initially to FIGS. 1-5, one sensor 10 of the present invention for detecting a presence of bacteria responsible for food borne illnesses may be described as including a housing 12 having a bore 14 fully extending through the housing and a pH sensitive material 16 carried within the bore. Foe one embodiment herein described, by way of example, the pH sensitive material 16 includes a pH indicator for providing a visual color change responsive to an increased level of carbon dioxide gas above an ambient level. As will be later described, various sensing materials may be carried within the sensor 10. For the indicator, herein described by way of example, a change in a gaseous bacterial metabolite concentration that is indicative of bacterial growth is detected, wherein the pH change is affected by a presence of the metabolite. The pH sensitive material 16 is carried within the bore 14 such that opposing first and second surfaces 18, 20 of the pH sensitive material 16 are exposed to an environment 22 within which the housing 12 is to be placed for monitoring and sensing the increased levels of carbon dioxide gas in the environment, as further illustrated with reference to FIG. 6.

With continued reference to FIGS. 1-5, a fastener 24 is carried by the housing 12 for freely and removably positioning the housing such that the first and second surfaces 18, 20 of the pH sensitive material 16 are in a spaced relation to any adjoining surfaces, such as those of food product 26 or a container wall 28 of a container 30 within the environment 22, thus permitting a free movement of the carbon dioxide gas thereabout and direct diffusion of the carbon dioxide gas onto and through the opposing first and second surfaces of the pH sensitive material, as illustrated with reference again to FIG. 6. Thus, gas diffusion on opposing exposed surfaces, surfaces 18, 20 of the pH sensitive material 16 is accomplished, rather than a sensitive surface on only one side, which is typically the case when a sensor is directly attached to a wall of the package material. A gap 32 or space between the pH sensitive material 16 and the packaging, the container wall 28 of a container 30, by way of example, or gap 34 between the pH sensitive material and a surface 27 of the food product 26, herein illustrated by way of example, permits gas to diffuse freely into the pH sensitive material, resulting in a faster detection time. By way of example with regard to the pH sensitive material 16, one such material may include mixture of Bromothymol Blue and Methyl Orange, which will go through a visual color change from green to orange as a result of an increased level of carbon dioxide gas diffusing through the pH sensitive material for increasing the hydrogen ion concentration and thus reducing the pH. In another example, the pH sensitive material 16 may comprise an edible pH indicator, extracted from plants, such as red cabbage or grape. Since these indicators tend to be unstable and last perhaps 24 hours, they may serve as a “one-day use only” sensor that changes color at the end of a 24 hour period regardless of food spoilage, and may be indicative of both bacterial load and freshness. One way to extend the life of the indicator is by incorporating up to 40% glucose or sucrose which slows down the rate of oxidation and breakdown. Yet further, the pH sensitive material 16 may comprise a gel, such as agar, and further may include an antifreeze agent, such as ethylene glycol or glycerol for preventing a freezing of any water component, thus allowing use with frozen foods.

By way of further example and with reference to FIG. 7, the sensor 10 may include the pH sensitive material 16 formed into first and second gas-permeable material portions 36, 38, each extending between the opposing first and second surfaces 18, 20. The first material portion 36 may comprise a buffered pH indicator having a reference color. The second material portion 38 may have a recognizable reference color at an initial pH level that changes to a recognizable caution or warning color at a predetermined pH level, wherein the warning color visually contrasts the reference color for alerting a user or consumer. Yet further, the first material portion 36 may include a time-temperature component, which will be discussed later in this section, while the second material portion 38 may include the pH sensitive material 16, each or both compared to a reference color of a reference material, or a reference color used for the housing 12.

By way of example and with reference again to FIG. 6, a thickness dimension 40 of the housing 12 may define the depth or thickness of the bore 14 and thus the thickness 42 or distance between the first and second opposing surfaces 18, 20 of the pH sensitive material 16 carried within the bore. With such definitions, one preferred ratio of the thickness 42 to effective width (a diameter fro the embodiment herein described) may be in a range of values from 0.003 to 0.3, for providing a desirable exposed surface area for a given thickness. By way of further example, the pH of the material may range from 7-10 in the ambient level carbon dioxide gas environment.

With reference to FIG. 8, the sensor 10 may include first and second gas permeable covers 42, 44 carried by the housing 12 for enclosing the pH sensitive material 16 within the bore 14. The covers 42, 44 may include gas permeable membranes or an impermeable material having holes 45 extending through the covers. The holes 45 may form a descriptive pattern representing a state (i.e. “S” for safe) of the pH sensitive material, by way of example. Further, the covers may have a predetermined color indicative of a pH level for the pH sensitive material, green for safe or orange for caution by way of example. Likewise, the housing may comprise a color representative of an initial color, visually indicating a safe condition, or a final color, indicating a potentially hazardous condition, for the pH sensitive material. By way of further example, the housing 12 may comprise a green color representative of the initial color. A color change from the green color to an orange color may result from the increased level of carbon dioxide gas.

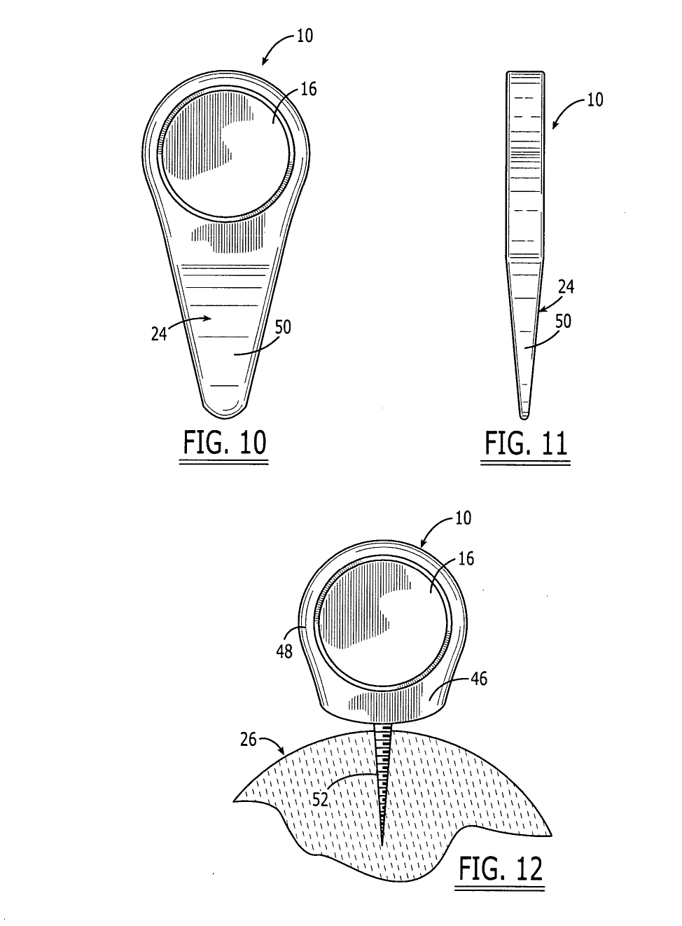

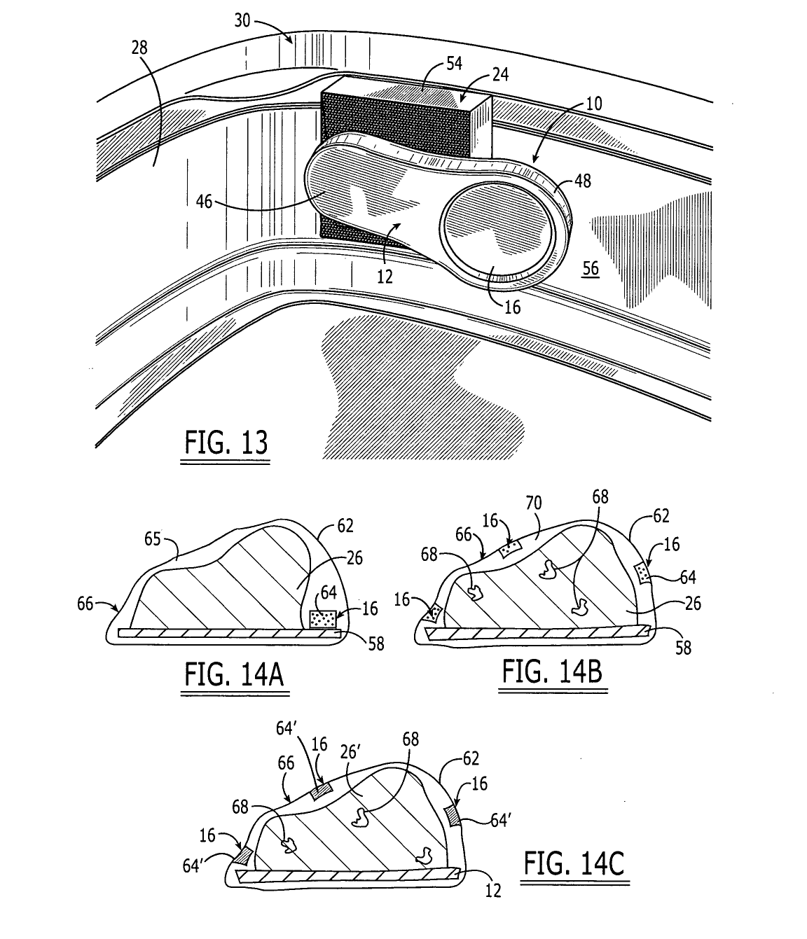

With reference again to FIGS. 1-5, one embodiment of the sensor 10, as herein described by way of example, may include the housing 12 having a handle portion 46 useful in handling the sensor by a user, and a sensing material portion 48 having the bore 14 for carrying the pH sensitive material 16. In one embodiment as illustrated with reference to FIGS. 10 and 11, embodiment, the fastener 24 may include a tapered portion 50 or as illustrated in another embodiment with reference to FIG. 12, may carry a pin 52 for piercing the food product 26 carried within the container 30, within which the food product 26 is to be stored. The fastener 24 may comprise an adhesive material carried by the housing 12, on the handle portion 46, by way of example. With reference again to FIGS. 1-5, the adhesive may be a Velcro material or an adhesive tape style material, as illustrated with reference again to FIGS. 6 and 8 for attaching the sensor 10 to the inside container 30 while placing the pH sensitive material 16 in a space relation to any nearby surfaces, such as the container wall 28, the food product 26, or general food product packaging elements, by way of example. With reference again to FIG. 6 and to FIG. 13, one preferred location for the pH sensitive material 16 is within a lower portion or lower one-half portion 56 of the container 30. Further, the housing 12 and the pH sensitive material 16 may be made of material safe for human consumption.

It is to be understood that sensor embodiments provide a change that may be based on absorbance (transmittance), fluorescence, or luminescence, the change being observable visually and/or using an optical instrument. Additionally, the pH sensitive material herein described may be chemically or physically attached to a solid support. For example, the sensor may be positioned within the food package carried by the packaging elements such as the wrapper or the tray that carries the food products. Alternatively, the pH sensitive material 16 or the sensor 10 may simply be placed within a package such as the container 30, herein described by way of example, attached to either the food product or to the container itself. Indeed, since carbon dioxide is heavier than air, it is sometimes preferable that the pH sensitive material 16 be located near a deep part of the container, such as the bottom half 56, as above described with reference to FIG. 6, by way of example.

By way of example, the sensor and methods herein described may be adapted to detect the presence of bacteria in shelf-life-sensitive packaged food products such as meats, poultry, fish, seafood, fruits, and vegetables using an on-board sensor comprising an indicator and housing. The sensor may be incorporated within a food package along with the food product, which is sealed to a substantially gas-tight level. In certain embodiments, it is believed advantageous to isolate the sensor from direct contact with the food product, and/or to detect the freshness of such packaged foods using a separate or incorporated sensor placed within the food packaging.

One sensor comprising an aqueous pH indicator, constructed to have an initial, pre-exposure pH opposite to an expected pH shift, is preferably isolated chemically or physically from the typically acidic environment present in a food sample, but unprotected from neutral gases. As bacteria multiply, metabolites are produced and diffuse into the pH indicator. The metabolite is sensed as a pH shift in the indicator, with a pH drop if the indicator is adapted to detect an acid, and a pH increase if the indicator is adapted to detect an alkaline substance. Typically, in order to detect CO₂, the pH sensitive material has a pH greater than pH7 and may be as high as pH 11, depending on the pKa.

An exemplary indicator comprises a material adapted to undergo a color change with a change in pH, such as Bromothymol Blue having an initial pH of 10.8 or phenol red, or cresol red, by way of example only. One embodiment of the invention includes a cocktail of Bromothymol Blue and methyl orange having an initial pH at about pH 7.2. Such an indicator changes from a green color to an orange color in the presence of CO₂ and thereby provides a universally accepted signal of safe and danger respectively (green/orange). An edible or nontoxic pH indicator may also be used, such as, but not limited to, extracts of red cabbage, turmeric, grape, or black carrot, obtained from a natural source such as a fruit or vegetable. Such indicators may have an initial pH of about 7.8. Tests have indicated that a sensor based on a pH indicator is capable of detecting a total pathogenic and non-pathogenic bacterial load equal to 1×10⁷ cfu/gram or less on food products, a level that has been identified by food safety opinion leaders as the maximum acceptable threshold for most food, for example.

Carbon dioxide may be used as a generic indicator of bacterial growth and to quantitatively estimate the level of bacterial contamination present in a sample. As is well known, when carbon dioxide comes into contact with an aqueous solution, the pH drops owing to the formation of carbonic acid, thus making pH an indicator of carbon dioxide concentration and, hence, of bacterial load. The embodiments herein described, by way of example, are capable of detecting a total pathogenic and non-pathogenic bacterial load at a level of at least 10⁷ cfu/g.

Another type of pH indicator measures the concentration of another metabolite comprising a volatile organic compound such as ammonia. In this embodiment the sensor comprises an aqueous solution having an initial pH in the acid range, for example, pH 4 by way of example, affected by the addition of an acid such as hydrochloric acid. As alkaline gases such as ammonia diffuse into the sensor, ammonia reacts with water to form ammonium hydroxide, which in turn raises the pH of the solution. As the pH level rises, a commensurate indicator change occurs, which, when detectable, is representative of food contamination.

A non-pH indicator may also be envisioned, wherein a bacterial metabolite diffuses into a sensor. This embodiment of one sensor comprises a chemical that precipitates out of solution in the presence of the metabolite. As an example, a calcium hydroxide sensor, in a concentration range of 0.0001-0.1 M, would form an observable precipitate of calcium carbonate in the presence of sufficient carbon dioxide.

In some embodiments, it may be desirable to incorporate a radiation shield into the sensor, to minimize photo-degradation of the indicator. For example, a colored dye may be incorporated to attenuate ultraviolet radiation, although this is not intended as a limitation.

A potential disadvantage of some gas sensors based upon sensing pH levels may include the possibility that, once the sensor is exposed to air, or if a pH change occurs within the food packaging, the sensor color could revert to a state wherein the food was indicated as being “safe,” even though a potentially unsafe bacterial load had been indicated previously. Thus it may be desirable in certain instances to incorporate a sensor wherein the changed state is nonreversible.

Such a difficulty could be overcome by using a sensor material that is unstable over a time period commensurate with a time over which the sensor is desired to operate. For example, anthrocyanine-based pH indicators derived from vegetables can break down via oxidation over a period spanning hours or days, which make their indication substantially irreversible. Alternatively, a precipitating embodiment could be used, either alone or in combination with one or more other sensors, wherein the precipitate does not dissipate, providing a substantially irreversible indicator.

Embodiments of the invention may include additives to prevent freezing of any water component of the sensor that may destroy or reduce pH-indicating activity. An antifreeze agent such as ethylene glycol or glycerol may be used to prevent freezing of the water component below 0° C. as in the case of food placed in a freezer.

With reference again to FIGS. 1 and 7, while a cylindrical, disk-like shape for the pH sensitive material 16 is herein illustrated, a plurality of shapes and configurations will be appreciated by one of skill in the art, including, but not limited to, disc-like, spherical, or rectangular. Disc-shaped elements are shown herein for several of the examples, since it is believed advantageous to provide as much surface area as possible when compared to a thickness of the material for enhancing gas diffusion into the sensor, to minimize state-changing time, and, therefore, to optimize sensitivity. Simply layering a film onto the interior surface of a container or packaging material limits the rate of gas diffusion to one side. Further, when a sensor is integrally formed with the package, it does not permit the user a desirable choice of including a sensor or not for a particular package.

With reference now to FIG. 14A-14C, a general operation of the pH sensitive material 16 is illustrated, wherein the material provided is gas-permeable and comprises an indicator that is adapted to detect a change in a gaseous bacterial metabolite concentration indicative of bacterial growth. A change is effected by a presence of the metabolite, and an observable change in the indicator is commensurate with a concentration of the metabolite.

As herein described by way of example, a tray 58 used to carry the food product 26 may be used to carry the pH sensitive material 16. In this embodiment, a unitary pH sensitive material 16 is positioned within an interior 60 of a sealing film 62 such as TPX, TPU, or PFA that are all permeable to CO₂ gas. It will be understood by one of skill in the art that a plurality of pH sensitive materials 16 could be used, and that packaging elements may also comprise, for example, a consumer-type sealable bag or container, such at the container 30 earlier described with reference to FIG. 6.

With continued reference to FIGS. 14A-14C, and by way of illustration, dotted shading 64 represents an initial state of the pH sensitive material, initially sensing a metabolite concentration of the air 65 trapped within the package 66 formed by the tray 58 and sealing film 62. With elapsed time and possible changes in storage temperature, bacterial colonies 68 begin to form on and within the food product 26, the bacterial colonies emitting a gaseous metabolite 70 that diffuses to the material 16 as illustrated with reference to FIG. 14B. The material 16 undergoes a chemical change indicative of the concentration of the metabolite 70. When the chemical change is sufficient to cause a detectable change, indicated by hatched shading 64′, a potential spoilage of the food product 26′ is indicated, as illustrated with to FIG. 14C. These parameters are dependent upon the characteristics of the sensing material 16, each calibrated so that a predetermined metabolite concentration limit is detectable.

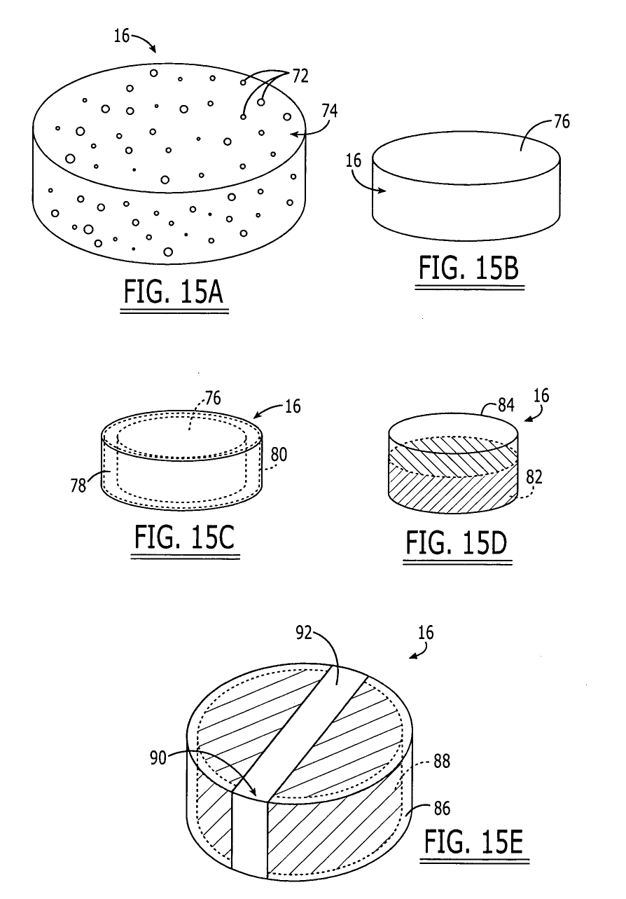

By way of further example, and with reference to FIG. 15A, one example of a sensing material 16 may be described as including an aqueous pH indicator 72 encapsulated within a silicone material 74. Silicone is substantially transparent, and is permeable to neutral gases but substantially impermeable to ions such as H⁺. When a metabolite such as carbon dioxide diffuses into the silicone material 74 and goes into solution in the pH indicator 72, the resulting pH change is reflected in an observable change, such as a color change, in the indicator. A housing 12 may be used to carry the pH sensitive material 16 as earlier described with reference to FIG.1, or freely carried within a package 66 as described with reference to FIG. 14A, by way of examples only. An exemplary form of the sensing material 16 comprises a thin disk, approximately 2.5 cm in diameter and 2-3 mm thick.

As illustrated with reference to FIG. 15B, another embodiment of the sensing material 16 may comprise an agar support 76 through which the indicator is substantially uniformly distributed. The aqueous indicator is mixed into the agar and allowed to cure. Agar is edible and safe for consumption. Yet further, the sensing material 16 may comprise agar or as described above that has been coated or covered with a proton-impermeable material 78 such as, a silicone material within a thin gas-permeable film 80 providing a barrier against charged particles while permitting neutral gas entry. Such may easily be employed for home/consumer use within sealable containers.

As illustrated with reference to FIG. 15D, another embodiment of the pH sensitive material 16 may comprise an indicator in solution 82 housed within a gas-permeable, but charged-particle-impermeable, clear container 84, such as a film or container. A support such as the housing 12, earlier described with reference to FIG. 1, may surround all or portion of the container 84, with such a structure providing two sided 18, 20 gas access. In addition, the fastener 24 may include the adhesive 54 earlier described with reference to FIG. 6 by way of example, applied to the handle portion 46 of the sensor 10 to permit the user to position the sensor inside a container, such as the container 30 above described.

Yet further, and as illustrated with reference to FIG. 15E, the pH sensitive material 16 may comprise an jacket 86 carrying a reference medium 88 and an indicator medium 90 positioned adjacent the reference medium. The reference medium 88 has a substantially constant state, e.g., a substantially immutable color that matches an initial state/color of the indicator medium 90. Thus when the indicator 90 experiences a change of state, the change will be evident from a comparison against the color of the reference 88. By way of example, the relative positioning of the indicator medium 90 and the reference medium 88 may provide a desirable formation, such as an icon indicative of spoilage, for example, a universal stop sign or other warning. In order to achieve such a relative positioning, the indicator medium 90 and the reference medium 88 comprise a unitary material, and the jacket 86 comprises a gas barrier such as transparent plastic positioned so as to leave at least a portion of the indicator medium 92 available to gas diffusion, using holes by way of example. Thus, only the indicator area 92 changes color under bacterial metabolite production, since the reference area is shielded therefrom. Alternatively, when a solid or semi-solid material such as silicone or agar is used to immobilize the pH indicator then the sensor may be comprised of two half portions, by way of example. One half portion may contain normal unbuffered pH indicator at an alkaline pH, while the other half portion contains a highly buffered indicator. Upon being brought in contact with carbon dioxide the unbuffered pH indicator would change color. However, the buffered indicator would remain the original color, a useful reference color.

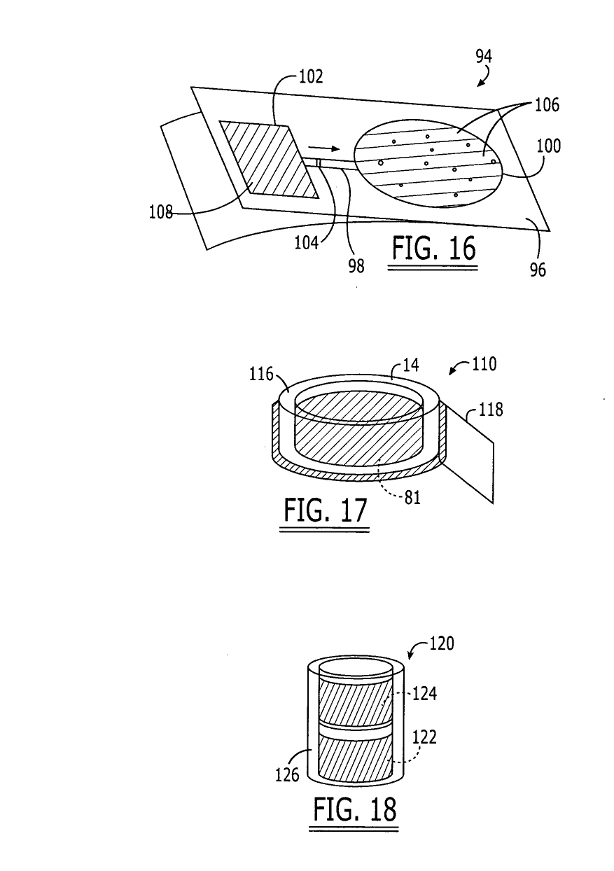

As illustrated with reference to FIG. 16, another embodiment of the present invention may include a sensor 94 may comprise a container support 96 and a fluid tube 98 affixed to the support. The gas-permeable sensor housing, which is positioned within an interior of food packaging, may comprise a first container 100 and a second container 102 fluidically isolated therefrom. In the example depicted in FIG. 2F, these containers 100,102 comprise “blisters” affixed to a substantially planar base of the container support 96 made, for example, of silicone or plastic, at least one of the blisters 100,102 being non-rigid. The fluid tube 98 extends between the blisters 100,102, but a frangible barrier 104 is positioned to block fluid access through the tube 98 unless and until a breaking of the frangible barrier 104 establishes fluid communication between the first 100 and the second 102 blister.

A pH indicator 106 in a substantially desiccated state is positioned within the first blister 100. In a hydrated state, the pH indicator 106 is adapted to detect a change in a gaseous bacterial metabolite concentration indicative of bacterial growth. Alternatively, the pH indicator may be kept in an aqueous acidic state (e.g., pH 3).

A hydrating/alkaline solution 108 is positioned within the second blister 102. The hydrating/alkaline solution 108 preferably has sufficient alkalinity (e.g., pH 10) that a mixture of the pH indicator 106 therewith results in an aqueous pH indicator having an initial pH in the alkaline range.

Thus, in storage, the first 100 and the second 102 blisters are fluidically isolated from each other, and, in use, the pressure is applied to either of the blisters to break the barrier 104, permitting the hydrating/alkaline solution 108 to mix with the pH indicator 106, and enabling the pH indicator 106 to perform its intended function. One advantage of retaining the pH indicator 106 in a desiccated or acidic state is increased shelf life, since some indicators, such as natural pH indicators, tend to be unstable under light exposure, oxidation, and extremes of temperature.

Another embodiment of a sensor 110, as illustrated with reference to FIG. 17, may comprise an aqueous solution 112 of indicator in silicone or agar, and as above described, carried within a gas-permeable, but charged-particle-impermeable, clear jacket 14, such as a film or container. The indicator solution 112 may be prepared at an alkaline pH, for example, pH 10, using, for example, sodium hydroxide. The jacket 114 is saturated with carbon dioxide 116, which lowers the pH, increasing the stability of the indicator solution 112. Activation is achieved by opening the jacket 114, such as by using a pull-tab 118. Exposure to air permits the carbon dioxide to escape, raising the pH of the indicator solution 112 back to approximately the initial pH, where the sensor 110 effectively functions.

As illustrated with reference to FIG. 18, another embodiment of a sensor 120, or the sensitive material 16 as earlier described with reference to FIG. 1, may comprise, in addition to a bacterial metabolite 122 as discussed above, a time-temperature integrative sensor 124 that tracks freshness, integrating temperature variations over time. Such a sensor 120 may also be incorporated into the sensor 94 of FIG. 16 or sensor 10 of FIG. 1. This sensor 120 may comprise a gas-permeable jacket 126 that is positioned within an interior of food packaging. Such a time-temperature integrator 124 provides an integrated temperature history experienced by the food packaging. By way of example, for many enzymes to function optimally, a moderate pH, an aqueous environment, and a temperature of approximately 37° C. are preferred. For every 10° C. reduction in temperature, enzyme activity is reduced by a factor of two. Additionally, enzymes tend to be relatively stable at 4° C.

In one embodiment, the time-temperature integrator 124 may comprise a substrate in solution that may be turned over by an enzyme to produce a color change. At 4° C. very little enzyme activity would occur, resulting in very little color change over the short term. However, at elevated temperatures enzyme activity would significantly increase, resulting in a substantial color change. Such a device would provide an integrated measurement of elevated time/temperature variations that would indicate a higher risk of food spoilage. The rate of reaction may be modified by careful selection of the appropriate enzyme temperature/activity profile. For example, an enzyme such as glucose oxidase may be used to catalyze glucose oxidation to form gluconic acid and hydrogen peroxide, and will, in the presence of an appropriate indicator, produce a color change. Hydrogen peroxide is a strong oxidizing agent that can be used to oxidize chromogenic indicators such as dianisidine producing a colorless to brown color change. The response of the integrator 124 to the degree of freshness may be adjusted by varying the chemical and/or physical components of the sensor 120. This in turn permits the tuning of the sensor to the requirements of a particular usage.

With continued reference to FIG. 18, another exemplary time-temperature integrator 124, positioned within a gas-permeable membrane 126, relies on the formation of an acid or carbon dioxide (which subsequently forms carbonic acid in solution). The detection of bacterial growth and time-temperature integration provides a user with two different pieces of information if the two sensors 122,124 operate independently. In this situation if either sensor 91,92 changes color, for example, the food product would be unacceptable for consumption. It is anticipated that these sensors 122,124 and those herein described, by way of example, will be configured as desired to meet individual needs by those skilled in the art now having the benefit of the teaching of et present invention.

Both the time-temperature environment and bacterial metabolite production directly and indirectly provide information regarding the freshness, quality, and safety of a perishable food product. Until the present invention a method of combining both indicators into a single, additive sensor has not been available. By combining both indicators into a single sensor or sensitive material 16, as earlier described and with reference again to FIG. 7, an overall estimate of freshness, quality, and safety for any given food product can be provided. Both indicators, which should act by experiencing pH changes in the same direction, contribute to form a more sensitive and accurate sensor.

In this example, a cocktail is prepared that consists of the bacterial carbon dioxide sensor components and the enzyme/substrate (time-temperature integrator) components combined with a pH indicator in a cocktail solution 128. This cocktail solution 128 is placed in a container 130 comprising, for example, silicone that is permeable to gases. The container 130 may then be adhered to the inner wall of the transparent film covering the food product, alternatively placed within the interior space of the packaging, or carried with the bore 14 as earlier described with reference to FIG. 1. The sensitive material 16, as earlier described, does not need to be in direct contact with the food, since any carbon dioxide produced by bacteria will permeate the entire container headspace. The carbon dioxide cocktail component consists of a weakly buffered solution. The time-temperature indicator cocktail comprises an enzyme/substrate combination comprising, for example, of a lipase enzyme and an ester substrate. A universal indicator that offers a large spectral change for a relatively small change in pH, e.g., Bromothymol Blue, is added to the cocktail.

Carbon dioxide produced by bacteria diffuses through the permeable container 130 into the cocktail solution 128, forms carbonic acid, and lowers the pH of the solution, resulting in an indicator color change. Depending upon the time-temperature environment, the enzyme turns over the ester substrate, producing fatty acid and alcohol. The fatty acid produced lowers the pH of the solution, also resulting in an indicator color change. Thus the sensor combines the output of both indicators in the same cocktail solution 128 to produce an additive color response. A reference 132 may also be incorporated in to the sensitive material to indicate that it is functioning as desired, and acts as a comparison reference.

By way of further example, if the embodiment of the sensor 94 described with reference to FIG. 16 is used, the combined pH indicator and enzyme/substrate components would be desiccated and positioned in the first blister 100, which would be advantageous in the case of unstable pH indicators comprising, for example, natural products.

By way of illustration, the data of Tables 1 and 2 were collected using a silicone sensor prepared as follows: A 5% w/v of Bromothymol Blue was prepared in aqueous solution. The pH was increased to pH 10 using concentrated sodium hydroxide. Agar was prepared by heating a block of agar to 55° C. 10% v/v of Bromothymol Blue was added to the agar and the solution was mixed to homogeneity. The agar was poured into 1-in.-diameter transparent containers to a depth of 2 mm and was allowed to cool at room temperature to form a deep blue flexible disk.

Chicken wings obtained from a local grocer were placed in 200-ml plastic sealable containers and incubated at 35° C. and 4° C. respectively. Agar indicators were prepared and placed adjacent to the chicken wings. The containers were then sealed. Drager tubes were used to determine the percent carbon dioxide present when the color changes. At 35° C. an indicator color change was first observed at 2.5 hours and a significant color change at 3 hours, comprising a blue to light green color change. The results provided in Table 1 indicate that approximately 1×10⁷ cfu/g of bacteria were detectable, and could be used as a means for a user to track the freshness and quality of shelf-life-dependent products. The data in Table 2 are provided as a control for chicken wings stored at 4° C.

TABLE 1
Effect of incubation of chicken at 35° C. on
biochemical and microbiological parameters.

| | Carbon Dioxide | Bacterial Concentration |
|---|---|---|
| Replicate | Concentration | (CFU/g) |
| 0-hours | BDL* | 6.2 × 10⁶ |
| 3 hours | | |
| Replicate 1 | 0.20% | 3.0 × 10⁷ |
| Replicate 2 | 0.17% | 2.9 × 10⁷ |
| Replicate 3 | 0.15% | 2.8 × 10⁷ |
| Average | 0.17% | 2.9 × 10⁷ |

*BDL = Below Detectable limits.

TABLE 2
Effect of incubation of chicken at 4° C. on
biochemical and microbiological parameters.

| | Carbon Dioxide | Bacterial Concentration |
|---|---|---|
| Replicate | Concentration | (CFU/g) |
| 0-hours | BDL* | 6.4 × 10⁴ |
| 48 hours | | |
| Replicate 1 | 1.0% | 4.3 × 10⁶ |
| Replicate 2 | 1.0% | 2.8 × 10⁶ |
| Replicate 3 | 0.6% | 4.2 × 10⁶ |
| Average | 0.87% | 3.8 × 10⁶ |
| Second batch of | | |
| chicken wings | | |
| 0-hours | BDL* | 7.8 × 10³ |
| 165 hours | | |
| Replicate 1 | 2.3% | 3.3 × 10⁷ |
| Replicate 2 | 3.5% | 4.4 × 10⁷ |
| Replicate 3 | 5.0% | 3.7 × 10⁷ |
| Average | 3.6% | 3.9 × 10⁷ |

*BDL = Below Detectable limits.
**NA = Not Applicable.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

Having now described the invention, the construction, the operation and use of preferred embodiments thereof, and the advantageous new and useful results obtained thereby, the new and useful constructions, and reasonable mechanical equivalents thereof obvious to those skilled in the art, are set forth in the appended claims.

That which is claimed is:

1. A sensor for detecting a presence of food borne bacteria, the sensor comprising:
a housing having a bore fully extending therethrough;
a pH sensitive material including a pH indicator for providing a visual color change responsive to an increased level of carbon dioxide gas above an ambient level thereof, the pH sensitive material carried within the bore and having opposing first and second surfaces exposed to an environment about the housing; and
a fastener carried by the housing for removably placing the opposing first and second surfaces of the material in a spaced relation to an adjoining surface, thus permitting a free movement of the carbon dioxide gas thereabout and direct diffusion of the carbon dioxide gas onto and through the opposing first and second surfaces of the pH sensitive material.

2. A sensor according to claim 1, wherein a color change from green to orange results from the increased level of carbon dioxide gas diffusing through the pH sensitive material for reducing a hydrogen ion concentration and thus reducing the pH.

3. A sensor according to claim 2, wherein the pH sensitive material comprises a mixture of Bromothymol Blue and Methyl Orange.

4. A sensor according to claim 1, wherein the pH sensitive material comprises a gel.

5. A sensor according to claim 4, wherein the gel comprises agar.

6. A sensor according to claim 5, wherein the agar is encapsulated within at least one of a permeable silicone, TPX, TPU, and PFA cover.

7. A sensor according to claim 1, wherein the pH sensitive material comprises an antifreeze agent.

8. A sensor according to claim 7, wherein the antifreeze agent comprises at least one of ethylene glycol and glycerol for preventing a freezing of any water component below 0° C.

9. A sensor according to claim 1, wherein the pH sensitive material comprises first and second material portions extending between the opposing first and second surfaces, the first material portion comprising a buffered pH indicator having a reference color, the second material portion having the reference color at an initial pH level and changing to a warning color at a predetermined pH level, the warning color visually contrasting the reference color.

10. A sensor according to claim 1, wherein a ratio of a thickness dimension to an effective width dimension of the pH sensitive material is in a range of values from 0.003 to 0.03.

11. A sensor according to claim 1, wherein the pH of the material ranges from 7-10 in the ambient level carbon dioxide gas environment.

12. A sensor according to claim 1, further comprising first and second gas permeable covers respectively enclosing the pH sensitive material and thus the opposing first and second surfaces within the bore, the permeable covers permitting the diffusion of the carbon dioxide gas therethrough.

13. A sensor according to claim 12, wherein the first and second covers comprise gas permeable membranes.

14. A sensor according to claim 12, wherein the gas permeable covers comprise a gas impermeable material having a plurality of holes extending therethrough.

15. A sensor according to claim 14, wherein the holes form a descriptive pattern representing a state of the pH sensitive material.

16. A sensor according to claim 15, wherein the cover comprises a predetermined color indicative of a pH level for the pH sensitive material.

17. A sensor according to claim 1, wherein the housing comprises a color representative of an initial color for the pH sensitive material.

18. A sensor according to claim 17, wherein the housing comprises a green color representative of the initial color, and wherein a color change from the green color to an orange color results from the increased level of carbon dioxide gas.

19. A sensor according to claim 18, wherein the pH sensitive material comprises a mixture of bromothymol blue and methyl orange.

20. A sensor according to claim 1, wherein the pH sensitive material is formed from an edible material and an edible pH indicator.

21. A sensor according to claim 20, wherein the edible pH indicator is formed from a food extract.

22. A sensor according to claim 21, wherein the food extract is processed from a food group consisting of cabbage, grapes, onions, berries, flowers, plums, and cherries.

23. A sensor according to claim 20, wherein the edible pH indicator comprises at least one of glucose and sucrose for reducing a rate of oxidation and breakdown thereof.

24. A sensor according to claim 20, wherein the edible pH indicator changes color within a predetermined time period regardless of bacterial growth and freshness of the food product.

25. A sensor according to claim 1, wherein the housing comprises a handle portion and a sensor portion, the sensor portion having the bore therein.

26. A sensor according to claim 25, wherein the fastener comprises a tapered portion formed within the handle portion for piercing a food product.

27. A sensor according to claim 1, wherein the fastener comprises an adhesive material carried thereby.

28. A sensor according to claim 27, wherein the adhesive material comprises Velcro.

29. A sensor according to claim 1, wherein the fastener comprises a pin carried by the housing for piercing a structure, wherein the structure includes at least one of a food product and a package.

30. A sensor according to claim 1, further comprising a container for carrying a food product therein, wherein the first and second surfaces of the pH sensitive material is carried within the container by the housing in a spaced relation to the food product and surfaces of the container.

31. A sensor according to claim 30, wherein the pH sensitive material is carried only within a lower one-half portion of the container.

32. A sensor for detecting a presence of bacteria in a perishable food product, the sensor comprising:
a housing having a bore extending fully therethrough;
a gas-permeable material carried within the bore so as to expose at least two opposing surfaces; and
a pH indicator carried by the gas-permeable material for detecting a change in a gaseous bacterial metabolite concentration indicative of bacterial growth, wherein a pH change results from the presence of the metabolite.

33. A sensor according to claim 32, further comprising a container dimensioned for receiving the perishable food product and the housing therein.

34. A sensor according to claim 32, wherein the pH indicator is responsive to an alkaline gas.

35. A sensor according to claim 34, wherein the alkaline gas comprises ammonia resulting from a protein breakdown of the food product.

36. A sensor according to claim 32, wherein the pH indicator is responsive to an acidic gas.

37. A sensor according to claim 36, wherein the acidic gas comprises carbon dioxide resulting from bacterial growth in the food product.

38. The sensor according to claim 32, wherein the pH indicator is adapted to exhibit a radiative change selected from a group consisting of absorbance, fluorescence, and luminescence.

39. The sensor according to claim 38, wherein the pH indicator undergoes a color change commensurate with the pH change.

40. The sensor according to claim 39, further comprising a reference color carried proximate the pH indicator for use in comparing the color change thereto.

41. The sensor according to claim 40, wherein a warning icon is formed by the color change.

42. The sensor according to claim 32, wherein the housing is temporarily attached to an interior of the container.

43. The sensor according to claim 32, wherein the pH indicator comprises an aqueous pH indicator, and wherein the housing further includes first and second gas permeable covers operable with the bore for securing the aqueous pH indicator therein.

44. The sensor according to claim 43, wherein the first and second covers are impermeable to charged particles.

45. The sensor according to claim 44, wherein the covers comprise a film material selected from a group consisting of TPX, PFA, and TPU film.

46. The sensor according to claim 32, wherein the gas permeable material comprises a substantially transparent silicone, and the pH indicator comprises an aqueous pH indicator encapsulated within the silicone.

47. The sensor according to claim 32, wherein the gas permeable material comprises a substantially transparent agar and the pH indicator comprises an aqueous pH indicator cured in a mixture with the agar.

48. The sensor according to claim 32, wherein the pH indicator comprises one of bromothymol blue, phenol red, and cresol red, the pH indicator having an initial color indicating an alkaline initial pH, the indicator turning a second color upon experiencing a decrease in pH.

49. The sensor according to claim 32, wherein the pH indicator comprises a mixture of bromothymol blue and methyl orange, the indicator having a green color indicating an initial alkaline pH of 7.2, the pH indicator turning an orange color upon experiencing a decrease in pH.

50. The sensor according to claim 32, wherein the housing is positioned within the container for placing the gas-permeable material in a spaced relation to interior walls thereof such that gas has access to both sides of the gas-permeable material thus permitting faster gas diffusion therethrough and a faster response time to a color change in the pH indicator.

51. The sensor according to claim 32, wherein at least a portion of the pH indicator is adapted to undergo a substantially irreversible change of state upon detecting the metabolite concentration change.

52. A sensor for detecting a presence of food borne bacteria, the sensor comprising:
a housing having a bore fully extending therethrough; and
a carbon dioxide indicator positioned within the bore for detecting bacterial growth, the indicator comprising an aqueous solution including calcium hydroxide, an infusion of carbon dioxide into the housing effecting a detectable calcium carbonate precipitate, wherein the indicator is exposed to an environment from opposing first and second ends of the bore.

53. A sensor according to claim 52, further comprising a fastener carried by the housing for placing the opposing first and second ends of the bore in a spaced relation to an adjoining surface, thus permitting a free movement of the carbon dioxide gas thereabout and direct diffusion of the carbon dioxide gas onto and through the carbon dioxide indicator.

54. A sensor according to claim 52, further comprising a substantially transparent silicone, wherein the carbon dioxide indicator an aqueous pH indicator encapsulated within the silicone.

55. A sensor according to claim 52, further comprising a substantially transparent film enclosing the carbon and substantially transparent container, the housing gas permeable and charged-particle impermeable.

56. The package recited in claim 52, wherein the carbon dioxide indicator comprises a substantially transparent agar and wherein the indicator is cured in a mixture with the agar.

57. A sensor for detecting a presence of bacteria in a perishable food product, the sensor comprising:
a gas-permeable material having at least two opposing surfaces exposed for a gas diffusion therethrough; and
a pH indicator carried by the gas-permeable material for detecting a change in a gaseous bacterial metabolite concentration indicative of bacterial growth, wherein a pH change results from the presence of the metabolite.

58. A sensor according to claim 57, further comprising a container dimensioned for receiving the perishable food product therein.

59. A sensor according to claim 57, wherein the pH indicator is responsive to an alkaline gas.

60. A sensor according to claim 59, wherein the alkaline gas comprises ammonia resulting from a protein breakdown of the food product.

61. A sensor according to claim 57, wherein the pH indicator is responsive to an acidic gas.

62. A sensor according to claim 61, wherein the acidic gas comprises carbon dioxide resulting from bacterial growth in the food product.

63. A sensor according to claim 57, wherein the gas permeable material and pH indicator are formed from an edible material.

64. A sensor according to claim 63, wherein the pH indicator is formed from a food extract.

65. A sensor according to claim 64, wherein the food extract is processed from a food group consisting of cabbage, grapes, onions, berries, flowers, plums, and cherries.

66. A sensor according to claim 64, wherein the pH indicator comprises at least one of glucose and sucrose for reducing a rate of oxidation and breakdown thereof.

67. A sensor according to claim 66, wherein the pH indicator changes color within a predetermined time period regardless of bacterial growth and freshness of the food product.

68. A method of detecting a presence of bacteria in a perishable food product comprising:
providing a container for receiving the food product therein;
placing the food product within the container;
providing a pH sensitive material including a pH indicator for providing a visual color change responsive to an increased level of carbon dioxide gas above an ambient level thereof; and
placing the pH sensitive material within the container, wherein at least two opposing surfaces of the pH sensitive material are in a spaced relation to the food product and walls of the container for permitting a free movement of the carbon dioxide gas thereabout and direct diffusion of the carbon dioxide gas onto and through the at least two opposing surfaces of the pH sensitive material.

69. The method according top claim 68, further comprising carrying the pH sensitive material by a housing.

70. The method according to claim 69, wherein the housing includes a bore extending therethrough, and wherein the pH sensitive material is carried therein.

71. The method according to claim 69, wherein the pH sensitive material placing comprises fastening the housing to at least one of the container and the food product.

72. The method according to claim 71, wherein the fastening comprises removably attaching the housing.

73. The method according to claim 68, further comprising:
monitoring the pH sensitive material for the visual color change; and
comparing a color resulting from the visual color change to a reference color.

74. The method according to claim 68, wherein a color change from green to orange results from the increased level of carbon dioxide gas diffusing through the pH sensitive material for reducing a hydrogen ion concentration and thus reducing the pH.

75. The method according to claim 68, wherein the pH sensitive material comprises a mixture of bromothymol blue and methyl orange.

76. The method according to claim 75, wherein a color change from green to orange results from the increased level of carbon dioxide gas diffusing through the pH sensitive material for reducing a hydrogen ion concentration and thus reducing the pH.

77. The method according to claim 76, further comprising:
removing the pH sensitive material from the container;
placing the pH sensitive material within another container; and
placing another food product in the other container, wherein at least two opposing surfaces of the pH sensitive material are in a spaced relation to the food product and walls of the other container for permitting a free movement of the carbon dioxide gas thereabout and direct diffusion of the carbon dioxide gas onto and through the at least two opposing surfaces of the pH sensitive material.

78. A method of detecting a presence of bacteria in a perishable food product comprising:
providing a pH sensitive material having at least two opposing surfaces, the pH sensitive material including a pH indicator for providing a visual color change responsive to an increased level of carbon dioxide gas above an ambient level thereof; and
exposing the pH sensitive material to the increased level of carbon dioxide for permitting the gas to be diffused onto and through the at least two opposing surfaces.

79. The method according to claim 78, further comprising placing the at least two opposing surfaces of the pH sensitive material in a spaced relation to the food product for permitting a free movement of the carbon dioxide gas thereabout and direct diffusion of the carbon dioxide gas onto and through the at least two opposing surfaces of the pH sensitive material.

80. The method according top claim 78, further comprising placing the food product and the pH sensitive material in a container for detecting the increased gas within the container.

81. The method according to claim 80, further comprising carrying the pH sensitive material only within a lower one-half portion of the container.

82. The method according to claim 78, further comprising adding an agent to the pH sensitive material for preventing a freezing of any water component therein below 0° C.

83. The method according to claim 78, further comprising:
adding a buffered pH indicator having a reference color indicative of an initial pH level as represented by an initial color; and wherein
the pH sensitive material includes the reference color at an initial pH level and changes to a warning color at a predetermined pH level, the warning color visually contrasting the reference color.

84. The method according to claim 78, further comprising:
a reference color indicative of an initial pH level as represented by an initial color; and wherein
the pH sensitive material includes the reference color at a first predetermined pH level and changes to a warning color at a second predetermined pH level, the warning color visually contrasting the reference color.

85. The method according to claim 84, further comprising:
providing a housing; and
carrying the pH sensitive material and the reference color by the housing.

86. The method according to claim 85, wherein the housing carries a color representative of the reference color.

87. The method according to claim 85, wherein the housing carries a color representative of the warning color.

88. The method according to claim 85, wherein the housing carries a green color, and wherein a color change from the green color to an orange color results from the increased level of carbon dioxide gas.

89. A method for detecting a presence of bacteria in a perishable food product, the method comprising:
carrying a pH indicator by a gas permeable material;
exposing at least two opposing surfaces of the material to a gaseous bacterial metabolite concentration indicative of bacterial growth in the perishable food product for diffusion of the gaseous bacterial metabolite therethrough; and
detecting a color change in the pH indicator resulting from the gaseous bacterial metabolite diffusion through the gas permeable material, wherein the change results from the presence of the metabolite.

90. A method according to claim 89, further comprising placing the gas-permeable material in a container having the food product carried therein.

91. A method according to claim 89, wherein the pH indicator is responsive to an alkaline gas.

92. A method according to claim 91, wherein the alkaline gas comprises ammonia resulting from a protein breakdown of the food product.

93. A method according to claim 89, wherein the pH indicator is responsive to an acidic gas.

94. A method according to claim 93, wherein the acidic gas comprises carbon dioxide resulting from bacterial growth in the food product.

95. A method according to claim 89, further comprising forming the gas permeable material and pH indicator from an edible material.

96. A method according to claim 95, wherein the pH indicator forming comprises processing a food extract.

97. A method according to claim 96, wherein the processing includes selecting from a food group consisting of cabbage, grapes, onions, berries, flowers, plums, and cherries.

98. A method according to claim 96, further comprising mixing at least one of glucose and sucrose with the pH indicator for reducing a rate of oxidation and breakdown thereof.

99. A method according to claim 98, wherein the pH indicator changes color within a predetermined time period regardless of bacterial growth and freshness of the food product.
